(19) United States
(12) Patent Application Publication
(10) Pub. No.: US 2010/0197805 A1
(43) Pub. Date: Aug. 5, 2010

(54) COMPOSITION FOR IMPROVING THE SURFACE APPEARANCE OF THE SKIN

(75) Inventor: Guillaume CASSIN, Villebon Sur Yvette (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/694,345

(22) Filed: Jan. 27, 2010

Related U.S. Application Data
(60) Provisional application No. 61/152,976, filed on Feb. 17, 2009.

(30) Foreign Application Priority Data
Jan. 27, 2009 (FR) 09 50491

(51) Int. Cl. A61K 47/30; A61Q 19/00

(52) U.S. Cl. 514/772.1

(57) ABSTRACT

The present invention relates to a cosmetic composition in the form of a paste, containing, in a physiologically acceptable medium, at least 10% by weight, relative to its total weight, of filler(s), at least one silicone polyamide block copolymer (PSPA) and at least one wax having a starting melting point of greater than or equal to 50° C. The present invention also relates to a cosmetic treatment method for improving the surface appearance of the skin, and in particular for reducing the visible and/or tactile irregularities of the skin, for instance the cutaneous microrelief, comprising at least the application of at least one composition as defined above to skin exhibiting said irregularities.

### REFERENCE TO PRIOR APPLICATIONS

This application claims priority to U.S. provisional application Ser. No. 61/152,976, filed Feb. 17, 2009; and to French patent application 09 50491, filed Jan. 27, 2009, both incorporated herein by reference.

### FIELD OF THE INVENTION

The present invention relates to a composition for improving the surface appearance of the skin, in particular of facial skin.

The composition according to the invention is more particularly for reducing the visible and/or tactile irregularities of the skin, for instance the cutaneous microrelief, or even the deep wrinkles of the skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

During the ageing process, the structure and functions of the skin become impaired. The main clinical signs observed are the appearance of wrinkles and fine lines associated with slackening of the skin. Those skilled in the art know that such slacking may be corrected immediately by applying a tensioning agent to the skin. The term “tensioning agent” is intended to mean a compound capable of having a tensioning effect, i.e. able to make the skin taut and, through this tensioning effect, to smooth out the skin and immediately reduce the wrinkles and fine lines, or even make them disappear.

To date, the use of numerous tensioning agents for treating wrinkles is known to those skilled in the art. Unfortunately, the effectiveness of such tensioning agents disappears over the course of the day, the tensioning film breaking up during the expressions made by the face. Furthermore, the effectiveness of these tensioning agents on the deepest wrinkles of the face is poor and not very long-lasting.

Consequently, there exists a great need for cosmetic compositions which make it possible to notably reduce even the deepest wrinkles, or even scars, on the face, throughout the day.

### DETAILED DESCRIPTION OF THE PREFERRED EMBODIMENTS

The present invention provides a novel composition for obtaining a comfortable deposit on the skin which gives long-lasting concealing of the wrinkles and imperfections of the face.

Thus, a main subject matter of the present invention is a composition in the form of a paste, containing, in a physiologically acceptable medium, at least 10% by weight, relative to its total weight, of filler(s), a silicone polyamide block copolymer (PSPA) and at least one wax having a starting melting point of greater than or equal to 50° C.

The subject matter of the present invention is also, according to another of its aspects, a cosmetic treatment method for improving the surface appearance of the skin, and in particular for reducing the visible and/or tactile irregularities of the skin, for instance the cutaneous microrelief, comprising at least the application of at least one composition in accordance with the invention to skin exhibiting said irregularities.

The method according to the invention advantageously results in a comfortable deposit on the skin, which gives long-lasting concealing of the visible and/or tactile irregularities of the skin.

It is also effective for treating wrinkles and fine lines, and most particularly the deep wrinkles of the skin, skin herein and above referring to human skin.

As specified above, the compositions according to the invention are in the form of a paste.

The term “paste” is herein intended to mean a composition, the viscosity of which can be measured, as opposed to the solid structure of a rod or stick, the viscosity of which cannot be measured.

The compositions according to the invention have characteristic rheological parameters, comprising the yield point, the elastic modulus and the viscous modulus. The destructuring yield or yield point τ_{O} stress of the compositions is defined as the pressure required to cause a macroscopic flow of the composition; it can be determined by scanning under stress, for example using a Haake applied stress CS150 rheometer at a temperature of 25° C., as described in example 1.

Moreover, the elastic modulus and the viscous modulus of the compositions may be measured for a stress frequency of 1 Hertz, in the “linear viscoelasticity” zone defined by the fact that the stress applied during the measurement is less than the destructuring yield stress for the composition.

In general, the compositions according to the invention are characterized by an elastic modulus of greater than 5000 Pascals, preferably greater than 10 000 Pascals, and even more preferably greater than 20 000 Pascals, and a tangent value (δ), given by the ratio of the viscous modulus to the elastic modulus, of less than 0.2, at 25° C. for a stress frequency of 1 Hertz.

The yield point of the compositions according to the invention is greater than 50 Pascals, preferably greater than 100 Pascals, and even more preferably greater than 500 Pascals.

For applied stresses below the yield point, the viscosity of the compositions according to the invention is greater than 100 000 Pa·s and preferably greater than 500 000 Pa·s.

The viscosity of the compositions according to the invention is greater than or equal to 5000 Pa·s for a shear rate equal to 10⁻³ s⁻¹, and preferably greater than 10 000 Pa·s, and in particular less than 10 000 000 Pa·s, the viscosity being measured at 25° C. with a Haake Rheostress RS 150 instrument in a cone-plate configuration under a stress.

With regard to their viscosity, the compositions according to the invention exhibit advantageous deformability, elasticity and manageability, making it possible to carry out modelling of the skin, in particular of the facial skin, which is therefore effective for reducing the cutaneous microrelief thereof.

For the purpose of the invention, the expression “visible and/or tactile irregularities of the skin” is intended to denote the signs of skin ageing, for instance wrinkles, in particular deep wrinkles, and fine lines, and also acne or chickenpox marks and scars.

The term “deep wrinkles” is intended to denote the wrinkles due to skin ageing, as opposed to “expression wrinkles” in particular induced, for their part, by dermocontraction of the facial muscles. It is the tensions responsible for expression wrinkles which, by pulling on the deep face of the dermis, have a tendency to become hollow and to form the deep wrinkles over time.

The present invention is more particularly dedicated to the cosmetic treatment of wrinkles, in particular of deep wrinkles of the skin, especially of deep wrinkles of the facial skin.

Silicone Polyamide Block Polymer

As indicated above, the compositions according to the invention comprise at least one silicone polyamide block polymer, also known as silicone polyamide.

The silicone polyamides of the composition are preferably solid at ambient temperature (25° C.) and atmospheric pressure (760 mmHg).

For the purpose of the invention, the term “polymer” is intended to mean a compound having at least 2 repeating units, preferably at least 3 repeating units, and even better still 10 repeating units.

The silicone polyamides of the composition of the invention may be polymers of the polyorganosiloxane type, for instance those described in documents U.S. Pat. No. 5,874,069, U.S. Pat. No. 5,919,441, U.S. Pat. No. 6,051,216 and U.S. Pat. No. 5,981,680. According to the invention, the silicone polymers may belong to the following two families:
(1) polyorganosiloxanes comprising at least two amide groups, these two groups being located in the polymer chain, and/or
(2) polyorganosiloxanes comprising at least two amide groups, these two groups being located on grafts or branches.

A) According to a first variant, the silicone polyamides are polyorganosiloxanes as defined above and in which the amide units are located in the polymer chain.

More particularly, the silicone polyamides may be polymers comprising at least one unit corresponding to general formula I:

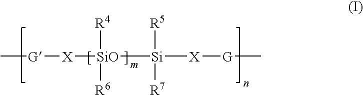

in which:

1) G′ represents C(O) when G represents —C(O)—NH—Y—NH—, and G′ represents —NH— when G represents —NH—C(O)—Y—C(O)—,

2) R⁴, R⁵, R⁶ and R⁷, which may be identical or different, represent a group chosen from:
saturated or unsaturated, C₁ to C₄₀ linear, branched or cyclic hydrocarbon-based groups which may contain one or more oxygen, sulphur and/or nitrogen atoms in their chain, and which may be partially or totally substituted with fluorine atoms,
C₆ to C₁₀ aryl groups optionally substituted with one or more C₁ to C₄ alkyl groups,
polyorganosiloxane chains optionally containing one or more oxygen, sulphur and/or nitrogen atoms,

3) the X, which may be identical or different, represent a C₁ to C₃₀ linear or branched alkylene diyl group, which may contain one or more oxygen and/or nitrogen atoms in its chain,

4) Y is a saturated or unsaturated, C₁ to C₅₀, arylalkylene, alkylarylene, cycloalkylene, arylene, or linear or branched alkylene divalent group which may comprise one or more oxygen, sulphur and/or nitrogen atoms and/or bear, as substituent, one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or

5) Y represents a group corresponding to the formula:

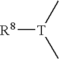

in which:
T represents a linear or branched, saturated or unsaturated, C₃ to C₂₄ trivalent or tetravalent hydrocarbon-based group optionally substituted with a polyorganosiloxane chain, and which may contain one or more atoms chosen from O, N and S, or T represents a trivalent atom chosen from N, P and Al, and
R⁸ represents a linear or branched C₁ to C₅₀ alkyl group, or a polyorganosiloxane chain, possibly comprising one or more ester, amide, urethane, thiocarbamate, urea, thiourea and/or sulphonamide groups, which may or may not be linked to another chain of the polymer,

6) n is an integer ranging from 2 to 500, preferably from 2 to 200, and m is an integer ranging from 50 to 1000, preferably from 50 to 700, and even better still from 50 to 200.

It will be noted that “m” corresponds to the average degree of polymerization of the silicone portion of the silicone polyamide.

According to one embodiment of the invention, 80% of the R⁴, R⁵, R⁶ and R⁷, of the polymer, are preferably chosen from methyl, ethyl, phenyl and 3,3,3-trifluoropropyl groups. According to another embodiment, 80% of the R⁴, R⁵, R⁶ and R⁷, of the polymer, are methyl groups.

According to the invention, Y may represent various divalent groups, optionally also comprising one or two valencies free to establish bonds with other units of the polymer or copolymer. Preferably, Y represents a group chosen from:

a) linear C₁ to C₂₀, preferably C₁ to C₁₀, alkylene groups,

b) C₃₀ to C₅₆ branched alkylene groups possibly comprising rings and unconjugated unsaturations,

c) C₅-C₆ cycloalkylene groups,

d) phenylene groups optionally substituted with one or more C₁ to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups, and

g) polyorganosiloxane chains of formula:

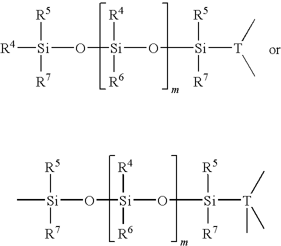

in which R⁴, R⁵, R⁶, R⁷, T and m are as defined above.

B) According to a second variant, the silicone polyamides suitable for the invention may be polymers comprising at least one unit corresponding to formula (II):

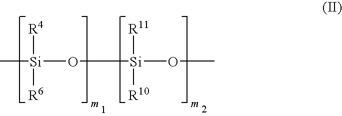

in which:
R⁴ and R⁶, which may be identical or different, are as defined above for formula (I),
R¹⁰ represents a group as defined above for R⁴ and R⁶, or represents the group of formula —X-G″—R¹² in which X are as defined above for formula (I) and R¹² represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,

and G″ represents —C(O)NH— and —HN—C(O)—;
R¹¹ represents the group of formula —X-G″—R¹² in which X, G″ and R¹² are as defined above,
m₁ is an integer ranging from 50 to 998, and
m₂ is an integer ranging from 2 to 500.

It will be noted that “m₁” corresponds to the average degree of polymerization of the silicone portion of the silicone polyamide.

According to the invention, the silicone polyamide may be a homopolymer, i.e. a polymer comprising several identical units, in particular units of formula (I) or of formula (II).

According to the invention, it is also possible to use a polymer formed from a copolymer comprising several different units of formula (I), i.e. a polymer in which at least one of R⁴, R⁵, R⁶, R⁷, X, G, Y, m and n is different in one of the units. The copolymer may also be formed from several units of formula (II), in which at least one of R⁴, R⁶, R¹⁰, R¹¹, m₁ and m₂ is different in at least one of the units.

It is also possible to use a polymer comprising at least one unit of formula (I) and at least one unit of formula (II), it being possible for the units of formula (I) and the units of formula (II) to be identical to or different from one another.

According to one variant of the invention, it is also possible to use a silicone polyamide comprising, in addition, at least one hydrocarbon-based unit comprising two groups capable of establishing hydrogen interactions, chosen from ester, amide, sulphonamide, carbamate, thiocarbamate, urea, urethane, thiourea, oxamido, guanidino and biguanidino groups, and combinations thereof.

These copolymers may be block polymers or grafted polymers.

In formulae (I) and (II), the alkylene group representing X or Y may optionally contain in its alkylene part at least one of the following components:

1) 1 to 5 amide, urea, urethane or carbamate groups,

2) a C₅ or C₆ cycloalkyl group, and

3) a phenylene group optionally substituted with 1 to 3 identical or different C₁ to C₃ alkyl groups.

In formulae (I) and (II), the alkylene groups may also be substituted with at least one component chosen from the group constituted of:
a hydroxyl group,
a C₃ to C₈ cycloalkyl group,
one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

In formula (I), Y may also represent:

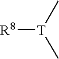

where R⁸ represents a polyorganosiloxane chain and T represents a group of formula:

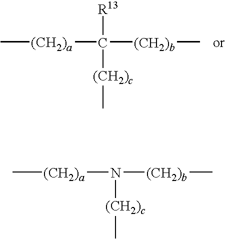

in which a, b and c are independently integers ranging from 1 to 10, and R¹³ is a hydrogen atom or a group such as those defined for R⁴, R⁵, R⁶ and R⁷.

In formulae (I) and (II), R⁴, R⁵, R⁶ and R⁷ preferably independently represent a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen above, the polymer may comprise identical or different units of formula (I) or (II).

Thus, the polymer may be a polyamide containing several units of formula (I) or (II) of different lengths, or a polyamide corresponding to formula (III):

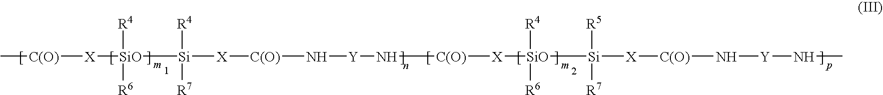

in which X, Y, n and R⁴ to R⁷ have the meanings given above, m₁ and m₂, which are different, are chosen within the range of from 1 to 1000, and p is an integer ranging from 2 to 300.

In this formula, the units may be structured in order to form either a block copolymer or a random copolymer or an alternating copolymer. In this copolymer, the units may be not only of different lengths, but also of different chemical structures, for example having different Y groups. In this case, the polymer may correspond to formula IV:

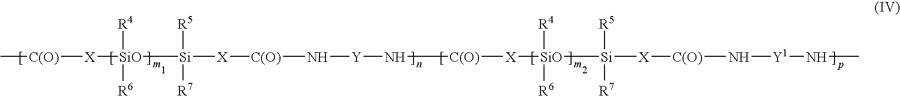

in which R⁴ to R⁷, X, Y, m₁, m₂, n and p have the meanings given above and Y¹ is different from Y but chosen from the groups defined for Y. As previously, the various units may be structured in order to form either a block copolymer or a random copolymer or an alternating copolymer.

In this first embodiment of the invention, the silicone polymer may also be formed from a grafted copolymer. Thus, the polyamide containing silicone units may be grafted and optionally crosslinked with silicone chains containing amide groups. Such polymers may be synthesized with trifunctional amines.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to one embodiment variant of the invention, a copolymer of silicone polyamide and of hydrocarbon-based polyamide, or a copolymer comprising units of formula (I) or (II) and hydrocarbon-based polyamide units, may be used. In this case, the silicone-polyamide units may be located at the ends of the hydrocarbon-based polyamide.

Advantageously, the composition comprises at least one polyamide/polydimethylsiloxane polymer, in particular a polymer of general formula (I) having an index m with a value of between 10 and 500, preferably between 10 and 300. Silicone polyamides with a degree of polymerization equal to 15, 100, 150 and 250 are found to be most particularly advantageous, and in particular that which has a DP of 100.

Advantageously, the silicone polyamide of formula (I) has a weight-average molecular mass ranging from 10 000 to 500 000 g/mol.

Preferably again, X and Y independently represent a group chosen from linear C₁ to C₂₀, preferably C₁ to C₁₀, alkylene groups.

By way of examples of a polymer that can be used, mention may be made of one of the silicone polyamides obtained in accordance with examples 1 to 3 of document U.S. Pat. No. 5,981,680, such as the product sold under the reference DC 2-8179 by Dow Corning.

The block copolymer of polydimethylsiloxane (DP=100) and of polyamide, the INCI name of which is Nylon 611/Dimethicone copolymer, and which is sold under the reference DC 2-8179 Silicone Polyamide, is most particularly suitable.

This polymer corresponds to the following formula:

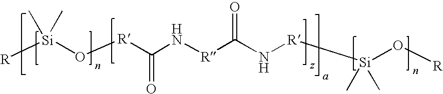

with n possibly being equal to 15, 100, 150 and 250, and preferably being equal to 100, and R, R′ and R″ representing saturated alkyl groups.

In general, a composition according to the invention may contain from 0.1% to 15% by weight, and more particularly from 0.5% to 10% by weight, of silicone polyamides.

As specified above, a composition according to the invention further comprises at least one, and preferably several, fillers.

Filler

The term “fillers” should be understood to mean inorganic or organic, colourless or white particles which are insoluble in the medium of the composition, irrespective of the temperature at which the composition is produced, and which do not colour the composition.

The fillers may be of any form, platelet-shaped, spherical, fibrous, hemispherical or oblong, irrespective of the crystallographic form (for example, lamellar, cubic, hexagonal, orthorhombic, etc.).

By way of illustration of these fillers, mention may be made of talc, mica, silica, kaolin, poly-β-alanine powder and polyethylene powder, tetrafluoroethylene polymer (Teflon®) powder, lauroyllysine, starch, boron nitride, hollow polymer microspheres such as polyvinylidene chloride/acrylonitrile microspheres, for instance Expancel® (Nobel Industrie), acrylic acid copolymers, silicone resin microbeads (for example Tospearls® from Toshiba), elastomer polyorganosiloxane particles, precipitated calcium carbonate, magnesium carbonate, magnesium hydrogen carbonate, hydroxyapatite, barium sulphate, aluminium oxides, polyurethane powders, composite fillers, hollow silica microspheres, and glass or ceramic microcapsules.

As specified above, a composition according to the invention may comprise fibres, in particular at least 5% by weight of fibres, relative to its total weight.

The term “fibre” should be understood to mean an object of length L and of diameter D such that L is greater than D, and preferably much greater than D, D being the diameter of the circle within which the cross section of the fibre falls. In particular, the L/D ratio (or shape factor) is chosen within the range of from 3.5 to 2500, preferably from 5 to 500, and better still from 5 to 150.

The fibres that can be used in the composition of the invention may be fibres of synthetic or natural, and inorganic or organic origin, and they may be flexible or rigid. They may be short or long, individual or organized, for example braided. They may have any shape, and may in particular have a circular or polygonal (square, hexagonal or octagonal) cross section, depending on the specific use envisaged. In particular, their ends are blunt and/or polished to prevent injury.

In particular, the fibres according to the invention preferably have a circular cross section.

In particular, the fibres have a length ranging from 1 μm to 10 mm, preferably from 0.1 mm to 5 mm, and better still from 0.1 mm to 3 mm. Their cross section is within a circle of diameter ranging from 2 nm to 500 μm, preferably ranging from 100 nm to 100 μm. The weight of the fibres is often given in denier or decitex.

The fibres may be those used in the manufacture of textiles, and in particular silk fibre, cotton fibre, wool fibre, flax fibre, cellulose fibre extracted, for example, from wood, from plants or from algae, polyamide fibre (Nylon®, in particular under the names Nylon 6=Polyamide 6; Nylon 6,6 or Nylon 66=Polyamide 6,6; Nylon 12=Polyamide 12), rayon fibre, viscose fibre, acetate fibre, in particular rayon acetate fibre, cellulose acetate fibre or silk acetate fibre, poly(p-phenyleneterephthalamide) fibre, acrylic polymer fibre, in particular polymethyl methacrylate fibre or poly(2-hydroxyethyl methacrylate) fibre, polyolefin fibre, and in particular polyethylene or polypropylene fibre, glass fibre, silica fibre, carbon fibre, in particular in graphite form, polytetrafluoroethylene (such as Teflon®) fibre, insoluble collagen fibre, polyester fibre, polyvinyl chloride fibre or polyvinylidene chloride fibre, polyvinyl alcohol fibre, polyacrylonitrile fibre, chitosan fibre, polyurethane fibre, polyethylene phthalate fibre, and fibres formed from a blend of polymers such as those mentioned above, for instance trilobed polyamide/polyester fibres, and mixtures of these fibres.

The fibres used in surgery may also be used, for instance the resorbable synthetic fibres prepared from glycolic acid and caprolactone (Monocryl from the company Johnson & Johnson); resorbable synthetic fibres of the lactic acid/glycolic acid copolymer type (Vicryl from the company Johnson & Johnson); terephthalic polyester fibres (Ethibond from the company Johnson & Johnson) and stainless steel threads (Acier from the company Johnson & Johnson).

Moreover, the fibres may be surface treated or untreated at the surface, and coated or uncoated. As coated fibres that can be used in the invention, mention may be made of polyamide fibres coated with copper sulphide to give an antistatic effect (for example, R-STAT fibres from the company Rhodia) or fibres coated with another polymer enabling a particular organization of the fibres (specific surface treatment) or a surface treatment inducing colour/hologram effects (Lurex fibre from the company Sildorex, for example).

The fibres that can be used in the composition according to the invention are preferably chosen from polyamide fibres, cellulose fibres and polyethylene fibres, and mixtures thereof. The length thereof may range from 0.1 to 5 mm, preferably from 0.25 to 1.6 mm, and the average diameter thereof may range from 5 to 50 μm.

Fibres chosen from Nylon 6 (or Polyamide 6), Nylon 6,6 or Nylon 66 (or Polyamide 6,6) and Nylon 12 (or Polyamide 12) fibres, and mixtures thereof, are most particularly suitable.

In particular, use may be made of the polyamide fibres sold by Etablissements P. Bonte under the name Polyamide 0.9 Dtex 0.3 mm (INCI name: Nylon 6,6), having an average diameter of 6 μm, a weight of approximately 0.9 dtex and a length ranging from 0.3 mm to 3 mm, alternatively the polyamide fibres sold under the name Fiberlon 931-D1-S by the company LCW, having a yarn count of approximately 0.9 dtex and a length of approximately 0.3 mm. Use may also be made of the Nylon-66 fibres, having a yarn count of approximately 2 dtex and a length of approximately 0.3 mm, sold under the name Polyamide brillante trilobée by the company Utexbel (INCI name: Nylon-66).

Use may also be made of cellulose (or rayon) fibres having an average diameter of 50 μm and a length ranging from 0.5 mm to 6 mm, such as those sold under the name Natural rayon flock fiber RC1BE-N003-M04 by the company Claremont Flock. Use may also be made of polyethylene fibres such as those sold under the name Shurt Stuff 13 099 F by the company Mini Fibers.

The fillers most particularly suitable for the invention are those chosen from:
porous silica microparticles, for instance Silica Beads SB150® and SB700® from Miyoshi, having an average size of 5 microns; Sunspheres® Series-H from Asahi Glass, for instance Sunspheres H33®, H51® and H53® having respective sizes of 3, 5 and 5 microns;
polytetrafluoroethylene (PTFE) powders, for instance PTFE Ceridust 9205F® from Clariant, having an average size of 8 microns;
silicone resin powders, for instance the Tospearl 145A® silicone resin from GE Silicone, having an average size of 4.5 microns;
hollow hemispherical silicone particles, for instance NLK 500, NLK 506 and NLK 510 from Takemoto Oil and Fat;
powders of acrylic copolymers, in particular of polymethyl (meth)acrylate (PMMA), for instance the PMMA particles Jurymer MBI® from Nihon Junyoki, having an average size of 8 microns, the hollow PMMA spheres for instance sold under the name Covabead LH85® by the company Wacker, and the vinylidene chloride/acrylonitrile/methylene methacrylate expanded microspheres sold under the name Expancel®;
polyethylene powders, especially comprising at least one ethylene/acrylic acid copolymer, and in particular constituted of ethylene/acrylic acid copolymers, for instance the AC540® polyethylene particles or else the Flobeads EA 209® particles from Sumitomo, having an average size of 10 microns;
crosslinked elastomeric organopolysiloxane powders coated with silicone resin, in particular with silsesquioxane resin, as described, for example, in U.S. Pat. No. 5,538,793. Such elastomer powders are in particular sold under the names KSP-100®, KSP-101®, KSP-102®, KSP-103®, KSP-104® and KSP-105® by the company Shin Etsu;
blends of crosslinked polydimethylsiloxane and of polydimethylsiloxane, for instance those sold under the names KSG-6® and KSG-16® by the company Shin Etsu;
composite powders of talc/titanium dioxide/alumina/silica, for instance those sold under the name Coverleaf AR-80® by the company Catalyst & Chemicals;
polyamide (Nylon®) powders, for instance the Nylon 12 particles of the Orgasol® type from Atofina, having an average size of 10 microns;
expanded powders such as hollow microspheres, and in particular the microspheres formed from a terpolymer of vinylidene chloride, acrylonitrile and methacrylate, for instance those sold under the name Expancel® by the company Kemanord Plast under the references 551 DE 12® (particle size of approximately 12 μm and density of 40 kg/m³), 551 DE 20® (particle size of approximately 30 μm and density of 65 kg/m³) and 551 DE 50® (particle size of approximately 40 μm);
the microspheres sold under the name Micropearl F 80 ED® by the company Matsumoto;
powders of natural organic materials, such as starch powders, in particular crosslinked or noncrosslinked maize, wheat or rice starch powders, such as the powders of starch crosslinked with octenylsuccinate anhydride, and in particular those sold under the name Dry-Flo® by the company National Starch;
and mixtures thereof.

According to one preferred embodiment, a composition according to the invention comprises at least one filler chosen from silica, porous silica microparticles, silicone resin powders, polyethylene powders, blends of crosslinked polydimethylsiloxane and of polydimethylsiloxane, and expanded powders, and mixtures thereof.

The amount of these fillers introduced depends of course on the desired effect, but they may generally represent from 10% to 65% by weight, preferably from 15% to 50% by weight, or even from 20% to 45% by weight, relative to the total weight of the composition.

Wax Having a Starting Melting Point of Greater than or Equal to 50° C.

As specified above, a composition according to the invention contains at least one wax having a starting melting point of greater than or equal to 50° C., and better still at least one wax of which the starting melting point is greater than or equal to 65° C.

In the present application, the term “starting melting point” is intended to mean the temperature at which a wax starts to melt. This temperature can be determined by DTA (differential thermal analysis), which allows the thermogram (or melting curve) of the wax under consideration to be obtained. The starting melting point corresponds to the temperature at which an appreciable change in the slope of the thermogram can be observed. The melting point itself represents the minimum point of said thermogram.

More specifically, such a wax may be chosen from Carnauba wax, certain polyethylene waxes and certain microcrystalline waxes, such as those sold under the name Microwax®, in particular the product sold under the name Microwax HW® by the company Paramelt.

The composition of the invention contains at least 3% by weight of such a wax, relative to its total weight.

The amount of wax(es) in the composition of the invention may more particularly range from 3% to 30%, and better still from 5% to 15% by weight, relative to the total weight of the composition. In order to efficiently incorporate the waxes and the fillers, it is advantageous to carry out the preparation of the composition or at least one step of this preparation, in particular the preparation of the oily phase or a step for preparing the oily phase, in a screw mixer-extruder subjected to a temperature gradient ranging from 100° C. to 20° C. It is in particular advantageous to mix the fillers, the waxes and at least one oil in a screw mixer-extruder subjected to a temperature gradient ranging from 100° C. to 20° C. Such a preparation is in particular described in applications EP 1 005 856, EP 1 005 857 and EP 1 013 267.

Interference Particles

A composition according to the invention may also advantageously comprise interference particles, for instance small pearlescent agents or interference pigments.

This is because such interference particles may together give the skin, coated with a film of a composition of the invention, a supplementary effect which lightens the skin, makes it uniform, or even camouflages the skin imperfections. Thus, by virtue of their presence, they advantageously make it possible to reinforce the visual perception of an improved surface appearance of the skin provided by the compositions under consideration according to the invention.

In particular, combined with the fillers, as defined above, the interference particles according to the invention may make it possible to obtain an effect of transparency such that the final effect provided on the skin has virtually no effect, or even no effect at all, on the natural flesh tone of the skin.

For the purpose of the present invention, the expression “interference particle” denotes a particle generally have a multilayer structure such that it allows the creation of a colour effect by interference of light rays, which diffract and scatter differently according to the nature of the layers. Thus, these particles may have colours that vary according to the angle of observation and the incidence of the light. The colour effects obtained are associated with the multilayer structure of these particles and are derived from the physical laws of thin film optics, as, for example, described in Pearl Lustre Pigments—Physical principles, properties, applications R. Maisch, M. Weigand. Verlag Moderne Industrie.

For the purpose of the present invention, the term “a multilayer structure” is intended to denote without distinction a structure formed from a substrate covered with a single layer or a structure formed from a substrate covered with at least two or even several consecutive layers.

The multilayer structure may thus comprise one, or even at least two, layer(s), each layer, optionally independently of the other layer(s), being made of at least one material chosen from the group constituted of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

Generally, the multilayer structure is of inorganic nature.

More particularly, the interference particles under consideration according to the invention may be interference pigments, or else pearlescent agents.

The interference particles according to the invention may have a volume-average size of generally less than 40 μm, especially ranging from 0.5 to 40 μm, more particularly less than 30 μm, especially less than 20 μm.

It is understood that the choice of these interference particles is made in such a way as to be, moreover, compatible with the demands in terms of filling required according to the invention. In general, these interference particles are present in an amount sufficient to obtain a homogeneous effect in terms of colouring while at the same time preserving the natural flesh tone of the skin and/or of the lips.

Pearlescent agents are most particularly suitable for the invention.

Pearlescent Agents

The term “pearlescent agents” should be understood to mean irridescent particles of any shape, in particular produced by certain molluscs in their shell, or which have been synthesized.

The natural or synthetic pearlescent agents may be monolayer or multilayer, in particular formed from a natural substrate based, inter alia, on mica and which is coated with one or more layers of metal oxide.

Thus, the pearlescent agents may be chosen from white pearlescent agents, such as mica coated with titanium, or with bismuth oxychloride, coloured pearlescent agents, such as titanium mica coated with iron oxides, with ferric blue, with chromium oxide or with an organic pigment of the abovementioned type, and also bismuth oxychloride-based pearlescent agents.

They may advantageously be chosen from mica/tin oxide/titanium oxide pearlescent agents, for instance those sold under the names Timiron Silk Blue®, Timiron Silk Red®, Timiron Silk Green®, Timiron Silk Gold® and Timiron Super Silk® proposed by the company Merck, and mica/iron oxide/titanium oxide pearlescent agents, for instance the Flamenco Satin Blue®, Flamenco Satin Red® and Flamenco Satin Violet® proposed by the company Engelhard and mixtures thereof.

The pearlescent agents may be present in a composition according to the invention in a content ranging from 0.1% to 50%, preferably from 0.1% to 40% by weight, and preferentially from 0.1% to 30% by weight, relative to the total weight of the composition.

More specifically, these pearlescent agents may represent from 0.1% to 15% by weight, more particularly from 0.1% to 7% by weight, and more particularly from 0.1% to 5% by weight, relative to the total weight of the composition.

In addition to the abovementioned compounds, a composition according to the invention may contain other organic materials of pigment or dye type, or else with a specific optical effect.

These supplementary materials may be present in the compositions according to the invention, in a content ranging from 0.1% to 15%, preferably from 0.5% to 12%, and preferentially from 1% to 10% by weight, relative to their total weight.

Material with Colour and/or Optical Effect

The term “dyes” should be understood to mean compounds which are generally organic and which are soluble in fatty substances such as oils or in an aqueous-alcoholic phase.

The fat-soluble dyes may be chosen from Sudan Red, DC Red 17, DC Green 6, β-carotene, Sudan Brown, DC Yellow 11, DC Violet 2, DC Orange 5 and quinoline yellow. The water-soluble dyes are, for example, beetroot juice and methylene blue.

The term “pigments” should be understood to mean inorganic or organic, white or coloured particles of any shape which are insoluble in the composition and intended to colour it.

Among the inorganic pigments, mention may be made of optionally surface-treated titanium dioxide, zirconium oxide or cerium oxide, and also zinc oxide, (black, yellow or red) iron oxide or chromium oxide, for instance those sold by the company Sunpuro under the reference PFX 5 Sunpuro Yellow, and Sunpuro Red iron oxide, manganese violet, ultramarine blue, chromium hydrate and ferric blue, metal powders such as aluminium powder and copper powder. The pigments may also be chosen from nanopigments of metal oxides, such as titanium dioxide, zinc oxide, iron oxide, zirconium oxide or cerium oxide, and mixtures thereof. The term “nanopigments” is intended to mean pigments having an average particle size ranging from 1 nm to 500 nm, and preferably ranging from 10 nm to 100 nm.

Among the organic pigments, mention may be made of carbon black, D & C pigments, and lakes, in particular lakes based on cochineal carmine, barium, strontium, calcium and aluminium.

Material with a Specific Optical Effect

An optical effect is different from a simple, conventional hue effect, i.e. a unified and stabilised effect of the kind produced by conventional colorants, such as, for example, monochromatic pigments. For the purpose of the invention, the term “stabilised” signifies absence of an effect of variability of colour with the angle of observation or else in response to a temperature change.

For example, the material capable of providing this effect may be chosen from metallic-glint particles, goniochromatic colouring agents, diffracting pigments, thermochromic agents and optical brighteners.

The metallic-glint particles that can be used in the invention are in particular chosen from:
particles of at least one metal and/or of at least one metal derivative,
particles comprising a single-substance or multi-substance, organic or inorganic substrate, at least partially coated with at least one metallic-glint layer comprising at least one metal and/or at least one metal derivative, and
mixtures of said particles.

Among the metals that may be present in said particles, mention may, for example, be made of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr, and mixtures or alloys thereof (for example bronzes and brasses) are preferred metals.

The term “metal derivatives” denotes compounds derived from metals, in particular oxides, fluorides, chlorides and sulphides.

By way of illustration of these particles, mention may be made of aluminium particles, such as those sold under the names Starbrite 1200 EAC® by the company Siberline and Metalure® by the company Eckart.

Mention may also be made of metal powders of copper or of alloy mixtures, such as the references 2844 sold by the company Radium Bronze, metal pigments, such as aluminium or bronze, for instance those sold under the name Rotosafe 700 from the company Eckart, the silica-coated aluminium particles sold under the name Visionaire Bright Silver from the company Eckart and the metal alloy particles such as silica-coated bronze (copper and zinc alloy) powders sold under the name Visionaire Bright Natural Gold from the company Eckart.

A composite pigment according to the invention may be composed in particular of particles comprising:
an inorganic core,
at least one at least partial coating with at least one organic colorant.

At least one binder may advantageously participate in the attachment of the organic colorant to the inorganic core. This binder may advantageously act without the formation of covalent bonds.

The particles of composite pigment may have varied shapes. These particles may especially be platelet-shaped or globular, in particular spherical, and may be hollow or solid. The term “platelet-shaped” denotes particles for which the ratio of the largest dimension to the thickness is greater than or equal to 5.

A composite pigment according to the invention may, for example, have a specific surface area of between 1 and 1000 m²/g, especially between 10 and 600 m²/g approximately, and in particular between 20 and 400 m²/g approximately. The specific surface area is the value measured by the BET method.

According to one particular embodiment of the invention, the inorganic core is a titanium oxide.

Titanium oxides, in particular TiO₂, iron oxides, in particular Fe₂O₃, cerium oxide, zinc oxide and aluminium oxide, and silicates, in particular aluminosilicates and borosilicates, are most particularly suitable as inorganic core.

The organic colorant may comprise, for example, organic pigments which may be chosen from the compounds below and mixtures thereof:
cochineal carmine,
organic pigments of azo, anthraquinone, indigoid, xanthene, pyrene, quinoline, triphenylmethane or fluorane dyes,
insoluble organic sodium, potassium, calcium, barium, aluminium, zirconium, strontium or titanium salts or lakes of acid dyes such as azo, anthraquinone, indigoid, xanthene, pyrene, quinoline, triphenylmethane or fluorane dyes, it being possible for these dyes to comprise at least one carboxylic or sulphonic acid group.

Among the organic pigments, mention may in particular be made of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 and FD&C Yellow No. 6.

According to one particular embodiment, the D&C Red No. 7 organic pigment is used.

According to another embodiment, the D&C Red No. 28 organic pigment is used.

According to another particular embodiment, the FD&C Yellow No. 5 organic pigment is used.

According to one particular embodiment, the organic FD&C Blue No. 1 aluminium lake is used.

According to another embodiment, the organic FD&C Yellow No. 5 aluminium lake is used.

According to one particular embodiment, the organic binder is a polymethylhydrogensiloxane.

By way of illustration of composite pigments of this type, mention may in particular be made of those composed as follows:
titanium dioxide (CI77891), FD&C Blue aluminium lake (CI42090) and polymethylhydrogensiloxane (58.1/40.7/1.2)
titanium dioxide (CI77891), D&C Red No. 7 (CI15850) and polymethylhydrogensiloxane (65.8/32.9/1.3)
titanium dioxide (CI77891), D&C Red No. 28 (CI45410) and polymethylhydrogensiloxane (65.8/32.9/1.3)
titanium dioxide (CI77891), FD&C Yellow 5 aluminium lake (CI191140) and polymethylhydrogensiloxane (65.8/32.9/1.3).

According to one alternative, a composite pigment suitable for the invention may also be composed of an inorganic core, in which is dispersed at least one organic or inorganic colorant, such as the pigments of Suzuki or Ercolano type.

By way of illustration of composite pigments suitable for the invention, mention may also be made of the pigments distributed under the name PC-LS-14 or PC-LS-19 by the company Miyoshikasei, and also the Rosso Er Colano pigments from the company Dolci-Colori.

The particles in question may also be particles comprising a glass substrate, such as those sold by the company Nippon Sheet Glass under the name Microglass Metashine.

The goniochromatic colouring agent may be chosen, for example, from multilayer interference structures and liquid-crystal colouring agents.

Examples of symmetrical multilayer interference structures that can be used in compositions prepared in accordance with the invention are, for example, the following structures: Al/SiO₂/Al/SiO₂/Al, pigments having this structure being sold by the company Dupont De Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by the company Merck (Darmstadt). By way of example, these pigments may be the pigments of silica/titanium oxide/tin oxide structure sold under the name Xirona Magic by the company Merck, the pigments of silica/brown iron oxide structure sold under the name Xirona Indian Summer by the company Merck and the pigments of silica/titanium oxide/mica/tin oxide structure sold under the name Xirona Carribean Blue by the company Merck. Mention may also be made of the Infinite Colors pigments from the company Shiseido. Depending on the thickness and the nature of the various layers, various effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

By way of example of pigments with a polymeric multilayer structure, mention may be made of those sold by the company 3M under the name Color Glitter.

Examples of liquid-crystal goniochromatic particles that may be used include those sold by the company Chenix, and also those sold under the name Helicone® HC by the company Wacker.

These materials may be present in a content ranging from 20% to 75% by weight, preferably from 20% to 50%, relative to the total weight of the composition.

Physiologically Acceptable Medium

As specified above, the compositions according to the invention comprise a physiologically acceptable medium, i.e. a nontoxic medium which can be applied to human keratin materials and which has a pleasant appearance, odour and feel.

The compositions according to the invention advantageously contain at least one liquid fatty phase formed from at least one oil.

The amount of oily phase in the composition of the invention generally ranges from 10% to 70%, and preferably from 20% to 50% by weight, relative to the total weight of the composition.

According to one embodiment variant, the compositions according to the invention may be in an anhydrous form.

For the purpose of the invention, the expression “anhydrous composition” denotes a composition which contains less than 2% by weight of water, or even less than 0.5% by water, relative to its total weight, and in particular a composition free of water.

As examples of oils that can be used in the composition according to the invention, mention may be made of:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides or else, for example, sunflower oil, maize oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil, shea butter oil;
synthetic esters and ethers, in particular of fatty acids, for instance oils of formulae R₁COOR₂ and R₁OR₂ in which R₁ represents the residue of a fatty acid containing from 8 to 29 carbon atoms and R₂ represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate, isostearyl isostearate, hydroxylated esters such as isostearyl lactate, octyl hydroxystearate, octyldodecyl hydroxystearate, diisostearyl malate, triisocetyl citrate, fatty alcohol heptanoates, octanoates and decanoates; polyol esters, such as propylene glycol dioctanoate, neopentyl glycol diheptanoate and diethylene glycol diisononanoate; and pentaerythritol esters, for instance pentaerythritol tetraisostearate;
linear or branched hydrocarbons of inorganic or synthetic origin, such as volatile or non-volatile liquid paraffins, and derivatives thereof, isohexadecane, isododecane, petroleum jelly, polydecenes, hydrogenated polyisobutene such as Parléam® oil;
natural or synthetic essential oils such as, for example, eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;
fatty alcohols containing from 8 to 26 carbon atoms, such as cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils such as those described in document JP-A-2-295912;
silicone oils, such as volatile or non-volatile polymethylsiloxanes (PDMSs) comprising a linear or cyclic silicone chain, which are liquid or pasty at ambient temperature, in particular cyclopolydimethylsiloxanes (cyclomethicones), such as cyclohexasiloxane and cyclopentasiloxane; polydimethylsiloxanes comprising pendent alkyl, alkoxy or phenyl groups or alkyl, alkoxy or phenyl groups at the end of the silicone chain, which groups contain from 2 to 24 carbon atoms; phenylated silicones, such as phenyl trimethicones, phenyl dimethicones, phenyltrimethylsiloxydiphenylsiloxanes, diphenyl dimethicones, diphenylmethyldiphenyltrisiloxanes, (2-phenylethyl)trimethyl-siloxysilicates and polymethylphenylsiloxanes;
and mixtures thereof.

The term “hydrocarbon-based oil” in the list of oils mentioned above is intended to mean any oil containing predominantly carbon and hydrogen atoms, and optionally ester, ether, fluoro, carboxylic acid and/or alcohol groups.

The compositions according to the invention may comprise a volatile oil.

For the purpose of the invention, the term “volatile oil” is intended to mean an oil capable of evaporating on contact with keratin materials in less than one hour, at ambient temperature and atmospheric pressure. The volatile oils of the invention are volatile cosmetic oils which are liquid at ambient temperature and have a non-zero vapour pressure, at ambient temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

As volatile oils, mention may be made, inter alia, of cyclic or linear silicones containing from 2 to 6 silicon atoms, such as cyclohexasiloxane, dodecamethylpentasiloxane, decamethyltetrasiloxane, butyltrisiloxane and ethyltrisiloxane. Use may also be made of branched hydrocarbons such as, for example, isododecane, and also volatile perfluoroalkanes such as dodecafluoropentane and tetradecafluorohexane, sold under the names PF 5050® and PF 5060® by the company 3M, and perfluoromorpholine derivatives, such as the 4-trifluoromethylperfluoromorpholine sold under the name PF 5052® by the company 3M.

The amount of oily phase present in the compositions according to the invention may range, for example, from 0.01% to 50% by weight, and preferably from 0.1% to 30% by weight, relative to the total weight of the composition.

The fatty phase of the compositions according to the invention may also comprise one or more solid fatty substances chosen in particular from pasty compounds and waxes other than the waxes defined above.

Waxes

For the purpose of the present invention, a wax is a lipophilic fatty compound which is solid at ambient temperature (25° C.), has a reversible solid/liquid change of state, has a melting point that is above approximately 45° C. (measured by DSC) and better still above 50° C. and that may be up to 200° C., and which, in the solid state, has an anisotropic crystalline organisation.

The waxes that are suitable for the invention are those generally used in the cosmetics and dermatological fields; they may be hydrocarbon-based, silicone-based and/or fluoro waxes, optionally comprising ester or hydroxyl functions.

As waxes that can be used in the composition of the invention, mention may, for example, be made of inorganic waxes such as microcrystalline waxes, paraffin, petroleum jelly, ozokerite, montan wax; waxes of animal origin, such as beeswax, lanolin and its derivatives; waxes of plant origin, such as candelilla wax, ouricury wax, Japan wax, cocoa butter, cork fibre wax or sugarcane wax; hydrogenated oils that are solid at 25° C.; fatty esters and glycerides that are solid at 25° C.; synthetic waxes such as polyethylene waxes and the waxes obtained by Fischer-Tropsch synthesis; silicone waxes; and mixtures thereof.

Pasty Compounds

The composition according to the invention may thus comprise a pasty compound other than the abovementioned hydrocarbon-based compounds. For example, such a compound may be chosen from polymeric or nonpolymeric silicone compounds and polymeric or nonpolymeric fluoro compounds.

The composition according to the invention may comprise, in addition to the abovementioned compounds, at least one structuring agent chosen from semicrystalline polymers, lipophilic gelling agents and mixtures thereof.

Thus, according to one embodiment, the composition according to the invention may comprise at least one gelling agent. The gelling agents that can be used in the compositions according to the invention may be polymeric or molecular, organic or inorganic lipophilic gelling agents.

As inorganic lipophilic gelling agent, mention may be made of clays that are optionally modified, such as hectorites modified with a C₁₀ to C₂₂ ammonium chloride, for instance the hectorite modified with distearyldimethylammonium chloride such as, for example, the product sold under the name Bentone 38V® by the company Elementis.

Mention may also be made of fumed silica which has been optionally hydrophobically surface-treated and the particle size of which is less than 1 μm.

They are, for example, sold under the references Aerosil R812® by the company Degussa, Cab-O-Sil TS-530® by the company Cabot,
Aerosil R972® and Aerosil R974® by the company Degussa, Cab-O-Sil TS-610® and Cab-O-Sil TS-720® by the company Cabot.

The hydrophobic fumed silica has in particular a particle size that may be nanometric to micrometric, for example ranging from approximately 5 to 200 nm.

The composition according to the invention may also comprise any usual cosmetic ingredient, which may be chosen in particular from antioxidants, fragrances, preservatives, neutralising agents, surfactants, sunscreens, sweeteners, vitamins, moisturisers and emollients, and mixtures thereof.

According to one advantageous embodiment, the compositions according to the invention may comprise at least one active agent.

Active Agent

A large diversity of active agents may be considered according to the invention.

They may in particular be chosen from: desquamating agents; anti-glycation agents; NO-synthase inhibitors; agents for stimulating the synthesis of dermal or epidermal macromolecules and/or for preventing their degradation; agents for stimulating fibroblast and/or keratinocyte proliferation; agents for stimulating or reducing keratinocyte differentiation; myorelaxants and/or dermal-decontracting agents; free-radical scavengers; and mixtures thereof.

According to one preferred variant of the invention, the active agent is an anti-ageing active agent, i.e. an agent having at least one preventive and/or curative effect on at least one cutaneous sign of ageing. These compounds are active agents that act on the epidermis and/or the dermis.

Examples of antiwrinkle active agents that can be used according to the invention are: retinol and derivatives thereof, such as retinyl palmitate; ascorbic acid and derivatives thereof, such as magnesium ascorbyl phosphate and ascorbyl glucoside; adenosine and derivatives thereof, in particular nonphosphated derivatives thereof; tocopherol and derivatives thereof, such as tocopherol acetate; nicotinic acid and precursors thereof, such as nicotinamide; ubiquinone; glutathione and precursors thereof, such as L-2-oxothiazolidine-4-carboxylic acid; C-glycoside compounds and derivatives thereof, in particular those described in application WO 02/051828; plant extracts, and in particular extracts of sea fennel and of olive leaf, and also plant proteins and hydrolysates thereof, such as rice or soybean protein hydrolysates; algal extracts and in particular extracts of laminaria; bacterial extracts; sapogenins, such as diosgenin and extracts of Dioscorea plants, in particular of wild yam, containing them; α-hydroxy acids; β-hydroxy acids, such as salicylic acid and 5-n-octanoylsalicylic acid; oligopeptides and pseudodipeptides and acyl derivatives thereof, in particular {2-[acetyl(3-trifluoromethyl-phenyl)amino]-3-methylbutyrylamino}acetic acid and the lipopeptides sold by the company Sederma under the trade names Matrixyl 500 and Matrixyl 3000; lycopene; manganese salts and magnesium salts, in particular gluconates; and mixtures thereof. Mention may also be made of vitamins such as, for example, vitamins B3 or PP, B5, E and K1.

As active agents that are most particularly suitable for the invention, mention may in particular be made of retinyl palmitate, tocopherol, tocopheryl acetate, lycopene, and the lipopeptides sold by the company Sederma under the trade names Matrixyl 500 and Matrixyl 3000, and C-glycoside derivatives, and in particular C-β-D-xylopyranoside-2-hydroxypropane.

Preferably, the anti-ageing active agent is chosen from adenosine and derivatives thereof, ascorbic acid and derivatives thereof, and C-glycosides and derivatives thereof such as C-α-D-xylopyranoside-2-hydroxypropane.

The active agents may also be active agents that act on greasy skin.

The expression “active agent for greasy skin” is intended to mean, in the context of the present invention, a compound which has, on its own, i.e. not requiring the involvement of an outside agent to activate it, a biological activity which may in particular be:
a desquamating activity, which makes it possible in particular to open comedones, and/or
an antimicrobial activity, in particular on P. acnes, and/or
a calming or anti-inflammatory activity, and/or
a sebum-regulating activity, and/or
an antioxidant activity, which prevents in particular the oxidation of squalene and the formation of comedones, and/or
a cicatrising activity, and/or
an astringent activity.

As examples of corresponding active agents that can be used in the compositions of the invention, mention may therefore in particular be made of desquamating agents, antimicrobial agents, calmatives, anti-inflammatories, sebum-regulating agents and antioxidants, and mixtures thereof.

The expression “sebum-regulating or anti-seborrhoeic” agents is intended to mean in particular agents capable of regulating sebaceous gland activity. They may in particular be retinoic acid, zinc salts, copper salts or aluminium salts, phthalimidoperoxyhexanoic acid, the tri(C₁₂-C₁₃)alkyl citrate sold under the name Cosmacol® ECI by the company Sasol; the tri(C₁₄-C₁₅)alkyl citrate sold under the name Cosmacol® ECL by the company Sasol or else 10-hydroxydecanoic acid.

The term “antimicrobial agents” is intended to mean agents which have effects on the specific flora of greasy skin, such as, for example, P. acnes.

These effects may be either bactericidal, or effects that act against bacterial adhesion, i.e. that prevent and/or reduce the adhesion of microorganisms, or effects that act on the biofilm of bacteria so as to prevent multiplication thereof.

As desquamating agents, mention may more particular be made of beta-hydroxy acids, such as 5-n-octanoyl salicylic acid; urea; glycolic acid, citric acid, lactic acid, tartaric acid, malic acid or mandelic acid; N-(2-hydroxyethyl)piperazine-N-(2-ethanesulphonic acid) (HEPES); extract of Saphora japonica; honey; N-acetylglucosamine; sodium methyl glycine diacetate, and mixtures thereof.

As calmatives, use may in particular be made of an agent chosen from an extract of rose, an extract of clove, dextran, as in Modulene® from Vincience, an extract of mint, such as Calmiskin® from Silab, a mixture of an extract of Nymphea alba and sodium palmitoylproline, for instance Seppicalm VG® from Seppic, aniseed derivatives, an extract of Paeonia suffruticosa and/or lactiflora, and mixtures thereof.

The term “antioxidants” is intended to mean agents which prevent the oxidation of squalene and the formation of comedones.

Mention may in particular be made of tocopherol and esters thereof, in particular tocopheryl acetate, BHT and BHA.

The amount of active agents depends of course on the nature of the active agent and on the desired effect, but said active agent generally represents from 0.01% to 10% by weight, preferably from 0.1% to 5% by weight, relative to the total weight of the composition.

Of course, those skilled in the art will take care to select the optional additional ingredients and/or the amount thereof in such a way that the advantageous properties of the composition according to the invention are not, or are not substantially, impaired by the envisaged addition.

Advantageously, the compositions according to the invention may be in the form of creams, granules, cohesive powders, or products cast as a stick or in a dish.

The composition according to the invention may be produced by known methods, generally used in the cosmetics or dermatological field.

A composition according to the invention may be prepared advantageously by using, for at least one step of the method, a mixer such as a roll mill comprising two rollers revolving in opposite directions, between which the paste passes, or a screw mixer-extruder. A screw mixer-extruder is preferably used.

The rest of the fat-soluble ingredients may then be mixed at a temperature of the order of 100° C. The ground material or the predispersed active agents may then be added to the oily phase.

The invention is illustrated in greater detail in the following examples, given by way of illustration and which are in no way limiting. The percentages are percentages by weight.

### Example 1

| | % by |
|---|---|
| | weight |
| MICROCRYSTALLINE WAX | 5.82 |
| (Microwax Hw ® sold by the company Paramelt) | |
| ISONONYL ISONONANOATE | 3.5 |
| SILICA & TITANIUM DIOXIDE & IRON OXIDES | 2.5 |
| (sold by Merck) | |
| Mixture of methyl, ethyl, propyl, butyl, isobutyl p- | 0.5 |
| hydroxybenzoates/2-phenoxyethanol | |
| (PHENONIP sold by Clariant) | |
| PROPYL P-HYDROXYBENZOATE | 0.07 |
| CYCLOPENTASILOXANE | 15 |
| (D5 sold by Dow Corning) | |
| POLYMETHYL SILSESQUIOXANE | 2 |
| (Tospearl145 ® sold by Toshiba) | |
| DIMETHICONE/VINYL DIMETHICONE CROSSPOLYMER | 12.45 |
| & DIMETHICONE | |
| (KSG 6 ® sold by Shin Etsu) | |
| DIMETHICONE/VINYL DIMETHICONE CROSSPOLYMER | 5 |
| & LAURETH-4 | |
| (DOW CORNING 9506 POWDER) | |
| METHYLSILANOL/SILICATE CROSS | 5.5 |
| (NLK 506 sold by Takemeto Oil and Fat) | |
| NYLON-611/DIMETHICONE COPOLYMER | 1 |
| (PSPA (DP100), sold under the reference DC | |
| 2-8179 silicone polyamide) | |
| MICA & TIN OXIDE & TITANIUM DIOXIDE | 2.5 |
| (Timiron silk red ® sold by Merck) | |
| HYDROGENATED POLYISOBUTENE | 22.39 |
| (Parleam ®) | |
| ETHYLENE/ACRYLIC ACID | 2.91 |
| (Polyethylene AC 540 ®, sold by Sumitomo) | |
| INCI name: COPOLYMERS ACRYLATES COPOLYMER & | 0.2 |
| ISOBUTANE | |
| (Expancel ®, sold by Nobel Industrie) | |
| SILICA | 2.66 |
| (Microporous silica SB700 ® sold by Miyoshi) | |
| Talc | 16 |

This composition is prepared using a BC21 mixer/extruder.

The composition of Example 1 is applied to the wrinkles, fine lines and imperfections of the cutaneous relief of a panel of women with mature skin. A significant decrease in the wrinkles and fine lines of these women is subsequently observed.

### Example 2

### Rheological Parameters

The composition of Example 1 was characterized by the following rheological parameters:
the destructuring yield or yield point τₒ stress was determined by scanning under stress using a Haake applied stress CS150 rheometer at a temperature of 25° C.;
the elastic modulus and the viscous modulus were measured for a stress frequency of 1 Hertz, in the “linear viscoelasticity” zone defined by the fact that the stress applied during the measurement is less than the destructuring yield stress for the composition.

The rheological parameters measured for the composition of Example 1 are reported in Table 1 below.

TABLE 1
Rheological parameters of the composition of Example 1

| | Example 1 |
|---|---|
| Elastic modulus G′ at 1 Hertz (Pa) | 106 000 |
| Viscous modulus G″ at 1 Hertz (Pa) | 17 000 |
| Yield point τ₀ (Pa) | 1160 |
| Viscosity (Pa) at 10⁻³ s⁻¹ | 1.2 × 10⁶ |

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. The term “mentioned” notes exemplary embodiments, and is not limiting to certain species. As used herein the words “a” and “an” and the like carry the meaning of “one or more.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

The invention compositions, etc. are preferably used by human subjects desirous of the benefits noted herein, subjects “in need of” these benefits. Such subjects are typically suffering from one or more of the conditions, symptoms, etc. addressed by the present invention, such as by self diagnosis or cosmetician or medical diagnosis, or are at recognized and appreciated risk of developing such conditions, etc. and who intentionally use the invention methods, compositions and combinations to treat, address, combat, prevent, etc. the effects of such conditions, etc. The application also clearly describes and supports the simple application of the invention composition on the skin and its integuments regardless of any purpose or intent.

1. A composition in the form of a paste, comprising, in a physiologically acceptable medium, at least 10% by weight, relative to its total weight, of filler(s), at least one silicone polyamide block copolymer (PSPA), and at least one wax having a starting melting point of greater than or equal to 50° C.

2. The composition according to claim 1, wherein the silicone polyamide block copolymer is at least a polymer comprising at least one unit corresponding to formula (I): 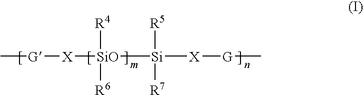
in which:
1) G′ represents C(O) when G represents —C(O)—NH—Y—NH—, and G′ represents —NH— when G represents —NH—C(O)—Y—C(O)—,
2) R⁴, R⁵, R⁶ and R⁷, which may be identical or different, represent a group chosen from:
saturated or unsaturated, C₁ to C₄₀ linear, branched or cyclic hydrocarbon-based groups which may contain one or more oxygen, sulphur and/or nitrogen atoms in their chain, and which may be partially or totally substituted with fluorine atoms,
C₆ to C₁₀ aryl groups optionally substituted with one or more C₁ to C₄ alkyl groups,
polyorganosiloxane chains optionally containing one or more oxygen, sulphur and/or nitrogen atoms,
3) the X, which may be identical or different, represent a C₁ to C₃₀ linear or branched alkylene diyl group, which may contain one or more oxygen and/or nitrogen atoms in its chain,
4) Y is a saturated or unsaturated, C₁ to C₅₀, arylalkylene, alkylarylene, cycloalkylene, arylene, or linear or branched alkylene divalent group which may comprise one or more oxygen, sulphur and/or nitrogen atoms and/or bear, as substituent, one of the following atoms or groups of atoms: fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or
5) Y represents a group corresponding to the formula: 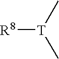
in which:
T represents a linear or branched, saturated or unsaturated, C₃ to C₂₄ trivalent or tetravalent hydrocarbon-based group optionally substituted with a polyorganosiloxane chain, and which may contain one or more atoms chosen from O, N and S, or T represents a trivalent atom chosen from N, P and Al, and
R⁸ represents a linear or branched C₁ to C₅₀ alkyl group, or a polyorganosiloxane chain, possibly comprising one or more ester, amide, urethane, thiocarbamate, urea, thiourea and/or sulphonamide groups, which may or may not be linked to another chain of the polymer,
6) n is an integer ranging from 2 to 500, and m is an integer ranging from 50 to 1000.

3. The composition according to claim 1, wherein the silicone polyamide block copolymer (PSPA) is at least a copolymer comprising at least one unit corresponding to formula (II): 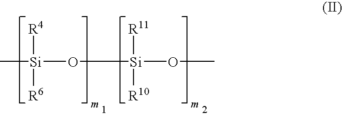
in which:
R⁴ and R⁶, which may be identical or different, represent a group chosen from:
saturated or unsaturated, C₁ to C₄₀ linear, branched or cyclic hydrocarbon-based groups which may contain one or more oxygen, sulphur and/or nitrogen atoms in their chain, and which may be partially or totally substituted with fluorine atoms,
C₆ to C₁₀ aryl groups optionally substituted with one or more C₁ to C₄ alkyl groups,
polyorganosiloxane chains optionally containing one or more oxygen, sulphur and/or nitrogen atoms,
R¹⁰ represents a group as defined in claim 2 for R⁴ and R⁶, or represents the group of formula —X-G″—R¹² in which X, which may be identical or different, represent a C₁ to C₃₀ linear or branched alkylene diyl group, which may contain one or more oxygen and/or nitrogen atoms in its chain, and R¹² represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups, and
G″ represents —C(O)NH— and —HN—C(O)—;
R¹¹ represents the group of formula —X-G″—R¹² in which X, G″ and R¹² are as defined above,
m₁ is an integer ranging from 50 to 998, and
m₂ is an integer ranging from 2 to 500.

4. The composition according to claim 1, wherein the silicone polyamide block copolymer (PSPA) is a silicone polyamide of formula (I) which has a weight-average molecular mass ranging from 10 000 to 500 000 g/mol.

5. The composition according to claim 1, wherein the silicone polyamide block copolymer (PSPA) corresponds to the following formula: 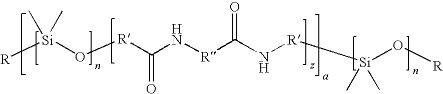
with n being equal to 15, 100, 150 or 250, and R, R′ and R″ representing saturated alkyl groups.

6. The composition according to claim 1, comprising, as filler, at least one compound chosen from:
porous silica microparticles,
polytetrafluoroethylene powders,
silicone resin powders,
hollow hemispherical silicone particles,
powders of acrylic copolymers,
polyethylene powders,
crosslinked elastomeric organopolysiloxane powders coated with silicone resin,
mixtures of crosslinked polydimethylsiloxane and of polydimethylsiloxane,
composite powders of talc/titanium dioxide/alumina/silica,
polyamide powders,
expanded powders such as hollow microspheres,
powders of natural organic materials, such as starch powders,
and mixtures thereof.

7. The composition according to claim 1, comprising from 15% to 50% by weight of filler(s) relative to the total weight of the composition.

8. The composition according to claim 1, further comprising interference particles.

9. The composition according to claim 8, wherein the interference particles are chosen from interference pigments and natural or synthetic pearlescent agents, which are monolayer or multilayer.

10. The composition according to claim 8, wherein the interference particles have a volume-average size of less than 40 μm.

11. The composition according to claim 1, comprising, as wax, at least one wax having a starting melting point of greater than or equal to 65° C.

12. The composition according to claim 1, comprising at least 3% by weight of wax(es) having a starting melting point of greater than or equal to 50° C.

13. The composition according to claim 1, wherein said wax with a starting melting point of greater than or equal to 50° C. is chosen from carnauba wax, polyethylene waxes and microcrystalline waxes.

14. The composition according to claim 1, having, at 25° C., a viscosity greater than or equal to 5000 Pa·s for a shear rate equal to 10⁻³ s⁻¹.

15. The composition according to claim 1, further comprising at least one oil.

16. The composition according to claim 15, comprising from 10% to 70% by weight of an oily phase relative to the total weight of the composition.

17. The composition according to claim 1, further comprising an anti-ageing active agent and/or an active agent that acts on greasy skin.

18. A method, comprising applying a composition according to claim 1 to human skin.

19. A cosmetic treatment method for improving the surface appearance of the skin, comprising applying a composition according to claim 1 to human skin in need thereof.

20. The cosmetic treatment method according to claim 19, wherein said method is a method for reducing the visible and/or tactile irregularities of the skin, comprising applying said composition to human skin in need thereof.
